(19) United States
(12) Patent Application Publication
(10) Pub. No.: US 2005/0266037 A1
(43) Pub. Date: Dec. 1, 2005

(54) Implantable biomaterial and method for the preparation thereof

(75) Inventors: Pei-Lin Mao, Singapore (SG); Yuri Shona Pek, Singapore (SG); Lihong Liu, Singapore (SG); Yuan Hong Yu, Singapore (SG)

Correspondence Address: SEED INTELLECTUAL PROPERTY LAW GROUP PLLC, 701 FIFTH AVE, SUITE 6300, SEATTLE, WA, 98104-7092, US

(73) Assignee: Agency for Science, Technology & Research, a comp. organized & existing under the laws of Singapore

(21) Appl. No.: 10/852,835

(22) Filed: May 25, 2004

(51) Int. Cl. A61F 2/28; A61K 35/32

(52) U.S. Cl. 424/423; 424/549

(57) ABSTRACT

The present invention relates to a method for the preparation of an implantable biomaterial comprising the steps of: obtaining bone tissue; boiling the bone tissue; and treating the bone tissue to remove the collagen. It also provides an implantable biomaterial prepared according to the process.

### FIELD OF THE INVENTION

The present invention relates to a new method for the preparation of implantable biomaterial (implantable bone grafting tissue). In particular, the present invention relates to a method for the preparation of anorganic implantable biomaterial and implantable biomaterial obtainable thereby.

### BACKGROUND OF THE INVENTION

The grafting of bone tissue is a technique used daily in most orthopedic surgery departments. The use of autografts, or bone obtained from a different part of the recipient's body, results in the need for additional surgery on the recipient, as well as donor site morbidity. However, the use of autograft is not completely satisfactory because it is painful for the patient and may involve a risk of complications at the donor site. Further, numerous operations require large amounts of bone tissue, which are incompatible with autografts. The use of bone allografts or xenografts involve implanting bone tissue from a donor into a recipient from the same species or different species, respectively. To perform the grafting, the bone tissues need to be treated so that they are clean and pure. The change in the mechanical properties of the bone tissues due to the treatment and the rejection due to immunogenecity are common problems for bone xenografts as well as allografts.

Allografts also have other drawbacks like the risks of infection of viruses and unsuccessfully recolonisation, while xenografts cause strong immune rejections. Most of the immune rejection is from proteins contained in the bone tissue, as well as cell debris/other elements in the medullary tissue. Slow/inaccurate recolonisation is due to proteins, mainly collagen, embedded in the extracellular matrix, thus leaving insufficient space for osteoblast cell penetration.

To solve the problem of rejection and recolonisation, extracting proteins from the bone tissue is the main step prior to implantation. However, most of the solvents used in the processing of animal bone are highly toxic. For example, U.S. Pat. No. 5,585,116 describes a protein extraction method using toxic solvents combined with a selective urea-based extraction agent. Further, these solvents are not easily removed completely through rinsing due to the high porosity of bone tissue. With reference to the processing of solvent-treated bone tissue, there are also other problems, in terms of complexity and high cost.

U.S. Pat. No. 5,725,579 and U.S. Pat. No. 6,217,614 describe a method for the preparation of bone organic matrix, mainly in collagen, comprising treating bone tissue with a fluid in supercritical state, for example, carbon dioxide (CO₂). This method therefore requires the use of a fluid in supercritical state. Further, this method requires a step of extraction of proteins by means of proteases. These steps of using fluid in supercritical state and protein extraction are quite costly processes.

Another suitable implant material is Bio-Oss Collagen from Ed. Geistlich Sohne A G Fur Chemische Industrie, which is a resorbable particulate bone mineral product comprising porous bone mineral nano-particles in a collagen matrix. The nano-particles are derived from natural bone and have an average diameter in the range of 0.1 to 10 μm. Bio-Oss Collagen is also described in U.S. Pat. No. 5,573,771. The problem with Bio-Oss is the difficulty of shaping the nano-particles into a tridimensional scaffold structure suitable as implantable bone grafting tissue. Other xenografts such as Osteograft/N (CeraMed, Lakewood, Colo.), and OsteoGen (Impladent, Hollisville, N.Y.) had been changed in the structures. In general, the common way to remove the organic matrix is by heating. However, when crystals are heated to temperatures of about 600° C., recrystallization takes place, and the crystals tend to grow, causing the structure of the material to change. Furthermore, some of the constituents are lost and some components are modified. As a result, all these xenografts tend to have less surface area, loss of some pores and reduced elasticity.

In view of the problems described above, there is a need in this field of technology for new, suitable, practicable and affordable implantable biomaterial.

### SUMMARY OF THE INVENTION

The present invention aims to alleviate all the aforementioned problems associated with processing, and provides an implantable biomaterial with high osteoinductivity, high osteoconductivity, biocompatibility, and comparable mechanical strength to natural bone. Most importantly, it is a very low cost and simple process.

According to a first aspect, the invention provides a method for the preparation of an implantable biomaterial comprising the steps of:
a) obtaining bone tissue;
b) boiling the bone tissue;
c) treating the bone tissue to remove the collagen.

In the method of the invention, the boiling step (b) may be carried out in water. For example, in distilled water.

In particular, the boiling step may be carried out to substantially disrupt the collagen and removing bone marrow and the extracellular matrix proteins (ECM).

A step of treating with ultrasound may be further applied to loosen and/or remove the remaining organic matrix.

The method according to the invention may comprise repeating the boiling step two or more times, optionally changing the water before the next step of boiling.

The method may optionally comprise a step of cleaning the bone tissue before boiling. The cleaning may be carried out according to any suitable method known in the art. For example, mechanically, by air or by means of a liquid. For example, the cleaning can be carried out by using water.

In the method of the invention, the step c) may comprise treating the bone tissue at a high temperature, for example at 210° C. so as to melt and denature the collagen, and further treating with a solvent to loosen the collagen. The solvent to loosen the collagen may be any non-toxic suitable solvent known in the art, preferably an alcohol. For example, ethanol. In particular, 70% ethanol.

The implantable biomaterial may also be treated with ultrasounds for maximizing the removal of collagen and/or for removing the disaggregated organic matrix (mainly collagen). The step may be carried out by treating with ultrasounds in the presence of an alcohol.

The method of the invention may further comprise a step d) of cutting the bone tissue into a predetermined shape. The bone tissue may be cut by using any suitable means known in the art, for example, by knife, scissors and/or cut up by means of a high-pressure water jet.

The method of the invention may further comprise a step e) of sterilising the cut bone before implantation.

The method of the invention may further comprise a step of packaging the implantable biomaterial.

The implantable biomaterial obtained or obtainable according to the present invention shows good osteoconductive capability due to no change of chemical and physical properties (that is, it facilitates a successful recolonisation of the grafts). In particular, the implantable biomaterial is an anorganic bone tissue free from the organic matrix.

The presence of collagen in the implantable biomaterial according to the invention has been assessed by using the SEM-EDX test. SEM-EDX detects the presence of sulfur (S), which is a component of collagen but is not present in the bone scaffold itself. No sulfur was detected in the implantable biomaterial of the invention by SEM-EDX. Accordingly, the invention provides an implantable biomaterial, which is free of collagen as detectable by SEM-EDX.

According to another aspect, the method of the invention comprises preparing an implantable biomaterial as described above and further, a step of implanting the biomaterial in a vertebrate.

In general, the implantation may be carried out for purposes of replacing and/or reconstructing a bone in a body, for example as a consequence of an injury or disease. It may also be used to replace and/or reconstruct periodontal defects and periodontal regeneration. The implantable biomaterial according to the invention may also be used to bone augmentation in general.

The method of the invention may further comprise a step of cultivating in vitro the implantable biomaterial prior to implantation. Further, the method may comprise a step of seeding the implantable biomaterial with the patient's own cells prior to implantation.

In particular, the composition of the implantable biomaterial obtained or obtainable according to any embodiment of the method of the invention has a of Ca/P ratio of 1.64.

In particular, the method of the invention does not comprise a step of treating the bone tissue with a fluid in supercritical state.

According to a further aspect, the invention provides an implantable biomaterial prepared according to any embodiment of the method of the invention. In particular, the composition of the implantable biomaterial has a Ca/P ratio of about 1.64.

The implantable biomaterial may be cultivated in vitro. For example, the implantable biomaterial may be seeded with the patient's own cells prior to implantation.

Accordingly, the invention also provides an in vitro cell culture comprising the implantable material prepared according to any embodiment of the method of the invention. The in vitro cell culture may comprise the implantable biomaterial seeded with the patient's own cells.

The implantable biomaterial can be shaped and sized in a way so as to make it suitable for a particular use. For example, it can have a size of 1 mm³ to 3 cm³. In particular, the size is 5 mm×5 mm (diameter/height).

### BRIEF DESCRIPTION OF THE FIGURES

FIG. 1: Animal bone prior to processing

FIG. 2: Cut bone scaffold, rinsed in ethanol, prior to implantation

FIG. 3: X-ray diffraction pattern analysis comparing the inorganic structure of anorganic porcine bone (A) and synthetic hydroxypatite (B).

FIG. 4: Overview of anorganic porcine bone structure from scanning electron micorgraph-energy dispersive X-ray (SEM-EDX).

FIG. 5: Component analysis with the ratio of Ca/P as 1.64.

FIG. 6A: The scaffold structures from SEM analysis of anorganic cancellous structure and dense structure (overall structure, ×35).

FIG. 6B: The scaffold structures from SEM analysis of anorganic dense structure (×3500).

FIG. 6C: The scaffold structures from SEM analysis of anorganic dense structure (×45000).

FIG. 6D: The scaffold structures from SEM analysis of anorganic cancellous structure (×2500).

FIG. 6E: The scaffold structures from SEM analysis of anorganic cancellous structure and (×8000).

FIG. 7A-D: SEM-DEX analysis of individual crystalline structures in the dense bone.

FIG. 8A-F: SEM-DEX analysis of individual crystalline structures in the spongiosa bone.

FIG. 9A: Morphology of normal osteoblast cell 3T3 before contacting with anorganic porcine bone.

FIG. 9B: Morphology of differentiated cell after contacting with anorganic porcine bone.

FIG. 10: Graph showing the ALP activity of normal osteoblast cell 3T3 (MC3T3) and differentiated cell (MC3T3/APB).

FIG. 11: Egg-like crystals with small dots.

FIG. 12: EDX result of crystalline formed in the solution containing 3T3 cells and APB.

FIG. 13A-F: TE micrographs of crystallines (A-C) formed in the solution containing 3T3 cells and APB for 2 weeks with components indicated by EDX (D-F).

FIG. 14A-C: TE micrographs of crystalline formed in the solution containing APB only for 2 weeks (A) with components indicated by EDX at different locations, site 1 (B) and site 2 (C).

FIG. 15A-B: TE micrograph of crystalline (A) formed in the solution containing C₂C₁₂ cells and APB for 2 weeks with components indicated by EDX (B).

FIG. 16A-B: SE micrograph of deposit on coverslip (A) and it's main components (B).

FIG. 17: XRD patterns of crystals in solution.

FIG. 18A-B: SE micrograph of BDS (A) and it's components detected by EDX (B).

### DETAILED DESCRIPTION OF THE INVENTION

The present invention aims to alleviate all the aforementioned problems associated with processing, and provides an implantable biomaterial with high osteoinductivity, osteoconductivity, biocompatibility, and comparable mechanical strength to a natural bone. Most importantly, it is a very low cost and simple process.

As used herein, the term “implantable biomaterial” is an “implantable bone grafting tissue”. Accordingly, for the purpose of the present application, the two terms may be used interchangeably. The implantable biomaterial of the invention is an anorganic bone granting tissue. With particular reference to the example, the term “anorganic porcine bone” (APB) has been used, which refers to the implantable biomaterial of the invention of porcine origin.

The present invention provides a new method for the preparation of an implantable biomaterial obtained from bone tissue.

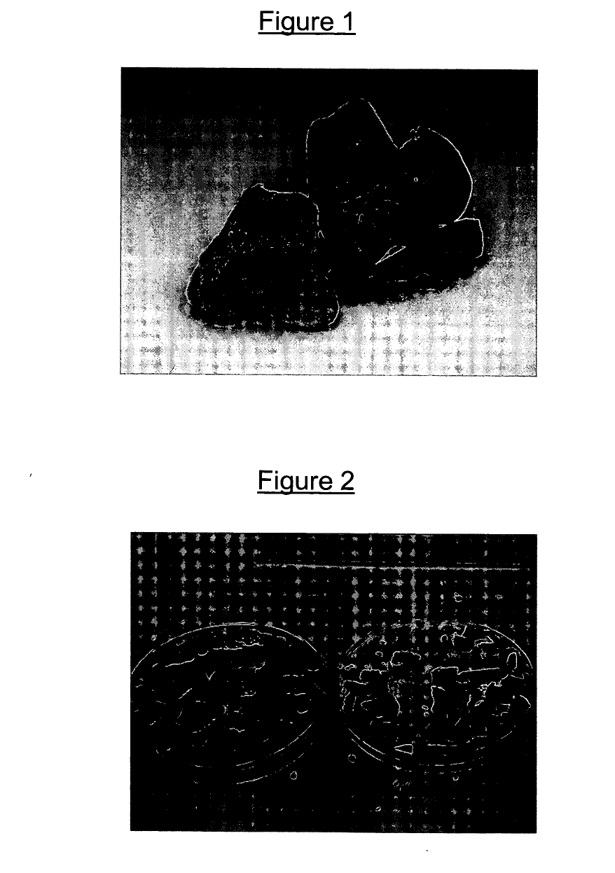

The implantable biomaterial may be prepared from the bone of animals that have been bred for consumption, which would otherwise have been disposed off after cooking (FIG. 1). These types of bone are therefore readily and cheaply available in large quantities. The bones can be obtained from any kind of suitable vertebrate animal. For example, from pigs, cows, and the like.

Bone is a type of connective tissue that forms the hard skeleton of most vertebrates. Bone is partly organic (cells and matrix) and partly inorganic (mineralized component). In any bone, the inorganic constituents are 65 to 70% on a dry weight basis and the organic constituents are 30 to 35% of a dry weight basis. Almost all of this inorganic substance (about 75%) is a compound called hydroxyapatite, which become deposited between collagen fibres. Collagen type I is the dominant collagen form in bone. Nearly 90-95% of the organic matrix (also indicated as organic material) is a substance called collagen, which is a fibrous protein. The rest, that is, 5-10%, comprises bone marrow of other non-collagen proteins. The non-collagen proteins comprises extracellular matrix proteins (ECM) and substances like chondroitin sulfate, keratin sulfate, and phospholipids. Accordingly, 30 to 35% of bone is collagen with a small fraction of other compounds. Collagen is embedded in a mucopolysaccharide ground substance. When bone becomes mineralized, the crystalline material becomes distributed regularly along the length of the collagen fibers. Bone marrow lies within the spaces between the trabeculae of all bones. It contains a variety of cells, including those active in haematopoiesis, fat cells and reticulum cells. Bone marrow has been defined as “ . . . is the soft material coming from the center of large bones, such as leg bones. This material, which is predominantly fat, is separated from the bone material by mechanical separation.” (Official Publication of American Feed Control Officials, 1997, page 191).

Five types of bone cells are found in bone skeletal tissue: osteoprogenitor cells, osteoblasts, osteocytes, osteoclasts and bone lining cells. Osteoblasts are involved in bone formation. Osteocytes arise from osteoblasts and subsequently become entrapped within the osseous tissue in lacunae and help maintain the bone matrix. Osteoclasts are multinucleated cells that are active in bone resorption.

According to one aspect, the present invention provides a method for the preparation of an implantable biomaterial comprising removing the organic matrix. In particular, the method of the invention provides a method for the preparation of an implantable biomaterial comprising the steps of:
a) obtaining bone tissue;
b) boiling the bone tissue;
c) treating the bone tissue to remove the collagen.

In the method of the invention, the boiling step (b) may be carried out in water. For example, in distilled water.

In particular, the boiling step may be carried out to substantially remove the organic matrix from the bones. The organic matrix, including collagen and non-collagen proteins is substantially de-aggregated and removed by the boiling step.

The method according to the invention may comprise repeating the boiling step two or more times, optionally changing the water before the next step of boiling. There is no particular limit to the time of boiling which can be chosen according to the particular bones of the particular animal. For example, the bones may be boiled from 20 minutes to 24 hours to remove the slurry including blood, bone marrow, lipid, and the remaining muscle. In particular the bones are boiled from 30 minutes to 6 hours, more in particular for 1-2 hours. According to a particular aspect, the method comprises a first step of boiling the bone tissue in water for 1 hour, and a second step comprising changing the water and continuing boiling for another one hour, and optionally repeating the steps a further two times.

The method may optionally comprise a step of cleaning the bone tissue before and/or after boiling. The cleaning may be carried out according to any suitable method known in the art. For example, mechanically, by air or by means of a liquid. Water, for example tap water, may be used to clean the bone tissue thoroughly.

In particular, after the boiling step, the bone tissue is cleaned thoroughly with water, for example deionized water.

The method may further comprise a step of drying the boiled bone tissue. The step of drying may be carried out by letting the bone tissue to dry or by heating. For example, by drying-heating in an oven. The heating in the oven may be carried out for a time suitable according to the kind and quantity of the bone tissue treated. For example, from 10 minutes to 2 hours, in particular 30 minutes to 1 hour. The temperature of the oven may be any suitable temperature according to the source from which the bone was obtained and to the type of bone, for example 150 to 300° C., in particular, 200 to 250° C. For example heating for 30 minutes to 2 hours at 210-220° C. Optionally, it is possible to change the position of the bone tissue to prevent overheating in certain areas.

After drying, the bone tissue may be treated in an autoclave for sterilisation. This step can be carried out for a suitable period of time and at a suitable temperature. For example, from 80 to 300° C., in particular, 100 to 200° C. The bone tissue can be autoclaved for 5 minutes to 2 hours at 100-200° C., for example for 15 minutes to 120-125° C.

In the method of the invention, the step c) comprises one or more steps carried out in order to remove the collagen. Any suitable method known in the art for removal of collagen may be used. For example, the step c) comprises treating the bone tissue at a high temperature (for example at a temperature of 150-300° C., in particular 200-250° C., more in particular 210-220° C., preferably 210° C. to melt and denature the collagen, and further treating with a solvent to dissolve the collagen. The solvent used to dissolve the collagen may be any non-toxic suitable solvent known in the art, for example a non-aqueous solvent, preferably an alcohol. For example, ethanol, in particular, 70% ethanol. Other non-aqueous solvents suitable for the purpose of the present invention for dissociating collagen and/or removing completely the residues of the organic matrix are: hydrazine, methanol, and/or guanidine.

The bone tissue may also be treated with ultrasound(s) for the maximal effect in removing the disaggregated (disrupted) and aggregated organic matrix (mainly collagen). The step may be carried out by treating with ultrasound(s) in the presence of an alcohol. The ultrasound treatment may be carried out according to the known standard protocol, for example for 30 minutes at 37° C. However, time and temperature may be varied according to the necessity. An anorganic bone tissue is obtained. In particular, the implantable material obtained or obtainable according to the invention is an anorganic bone tissue completely free from organic matrix.

The presence of collagen in the implantable biomaterial according to the invention has been assessed by using the SEM-EDX test. SEM-EDX detects the presence of sulfur (S), which is a major component of collagen but is not present in the bone scaffold itself. Through the analysis of SEM-EDX in many areas of spongiosa and cortical bones no sulfur was detected in any area of the anorganic bone, as shown in Tables 1 to 10. Accordingly, the invention provides an implantable biomaterial, which is free of collagen as detectable by SEM-EDX. In particular, the step (c) of the method of the invention comprises treating the bone tissue to remove the collagen so that the bone tissue is free of collagen as detectable by SEM-EDX.

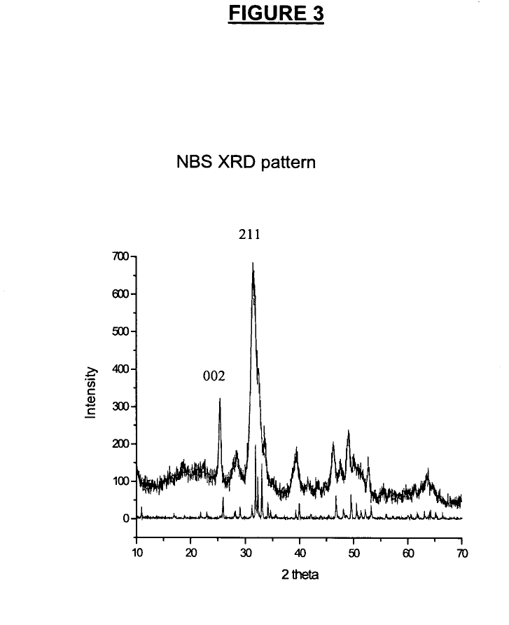

The method of the invention may further comprise a step d) of cutting the bone tissue into a predetermined shape (FIG. 2). The bone tissue may be cut by using any suitable means known in the art, for example, by knife, scissors and/or cut up by means of a high pressure water jet. The water-jet cutting allows accuracy of up to 10 μm, and it is carried out using a jet of pure water, thus avoiding any risk of contamination from a cutting tool, and further it can be used for mass production purposes. The shape of the pieces of the bone tissue can be of any suitable shape for implantation, for example it may be a right-angled parallelepiped, a cylinder, plug-shaped, or the like. The implantable biomaterial can be sized according to the size for the particular use. For example, it can have a size of 1 mm³ to 3 cm³. The suitable size and/or shape can however be selected according to the size of the trauma. A preferred size may be, for example, 5 mm×5 mm (diameter/height) (Martin, I., et al., J. Orthopaedic Res, 16:181-189, 1998; Wei Tan, B. S., et al,. Tissue Engineering, 7:203-210, 2001).

The method of the invention may further comprise a step e) of sterilising the cut bone before implantation.

A step consisting of the dehydration and disinfection of the bone tissue may optionally be carried out at any stage of the method of the invention. This step may be carried out by passing it through several successive baths of increasingly concentrated ethanol, for example 70%, 95% and 100%. Because ethanol is an excellent virucidin, it makes it possible to simultaneously dehydrate the tissue and increase the safety of the biomaterial with regards to infection. Drying in a ventilated oven at a suitable temperature, for example 30-80° C., preferably 30-60° C. may complete this step.

The method of the invention may further comprise a step of packaging the implantable biomaterial. After being packaged, the bone tissue may be then subjected to sterilisation. This sterilisation may be carried out according to any method known in the art, for example by irradiation, either by beta particles or by gamma rays (25 k Gray).

According to a particular embodiment, the method according to the invention comprises the steps of:
obtaining the bone tissue;
cleaning the bone tissue with water;
boiling the bone tissue in water;
cleaning the boiled bone tissue;
drying the bone tissue;
treating the bone tissue at a high temperature to melt and denature collagen;
treating the bone tissue with a solvent to sterilize and/or loose aggregated organic matrix;
allowing the solvent to evaporate from the bone tissue;
optionally, further treating the bone tissue with ultrasound(s) for completely removing the aggregated organic matrix (mainly collagen).

More in particular, the method of the invention comprises:
obtaining the bone tissue;
cleaning the bone tissue with tap water thoroughly;
boiling the bone tissue in the water for 1 hour, consistently removing the slurry;
changing the water and continuing to boil for another 1 hour, repeating for a further two times;
cleaning the bone tissue thoroughly with deionized water;
oven-drying for 30 minutes at 220° C.;
autoclaving at 121° C. for 15 minutes;
oven-drying for 2 hours at a temperature high enough to melt and denature collagen;
immersing the bone in ethanol for 2 days to further loose the collagen;
allowing the ethanol to evaporate from the bone by air-drying;
cutting the bone tissue according to the predetermined shape;
rinsing the cut bone in ethanol to sterilize it before implantation.

The implantable biomaterial obtained or obtainable according to the present invention shows a good osteoconductive capability (that is, it facilitates a successful recolonisation of the grafts).

The composition of the implantable biomaterial obtained or obtainable according to any embodiment of the invention has a Ca/P ratio of 1.64, which is slightly lower than the natural bone (than human bone as shown in Example 1).

U.S. Pat. No. 5,725,579 and U.S. Pat. No. 6,217,614 (both herein incorporated by reference) describe a process for preparing an implantable bone organic matrix, mainly in collagen, with improved mechanical strength comprising the step of treating the bone tissue with a fluid in supercritical state adapted to obtain a tissue containing less than 2% fat on average. Further, the process of the prior art comprises the step where the bone tissue, which has been treated with the fluid in supercritical state is subjected to an additional conventional process involving chemical or enzymatic treatment to extract specific proteins. The additional chemical treatment may be carried out using hydrogen peroxide, while the enzymatic treatment may be effected by means of a protease. This additional treatment ensures more effective extraction of the proteins from the bone tissue and accordingly decreases the risk of rejection of the bone tissue which has been treated in this way.

On the contrary, the implantable biomaterial obtained according to the method of the invention does not have a strength greater than a natural bone, but is has a strength comparable to that of a natural bone. Further, the method according to the invention does not require a step of treating with a fluid in supercritical state and further does not require the removal of proteins by protease. In fact, the method of the invention comprises removing the organic matrix within the bone matrix, which consists of collagen fibres (about 90-95% of the organic substance, also termed as organic matrix) and ground substance. The hardness of the matrix is due to its content of inorganic salts (hydroxyapatite; about 75% of the dry weight of bone), which become deposited between collagen fibres. The organic matrix is substantially removed by the step of boiling the bone tissue. The remaining of the organic matrix substantially comprising collagen is removed using any suitable method known in the art, for example by treating the bone tissue with a suitable solvent, in particular a non-aqueous solvent, for example an alcohol. Accordingly, the method of the invention comprises dissolving the collagen with a solvent, which may be ethanol (in particular, 70% ethanol), hydrazine, methanol, and/or guanidine hydrochloride. However, other suitable non-aqueous solvents known in the art may also be used.

Accordingly, the method of the present invention does not comprise a step of treating the bone tissue with a fluid in supercritical state. Further, the method according to the invention does not comprise a step of removing proteins with a protease or any other enzymatic treatment.

According to another aspect, the method of the invention comprises preparing an implantable biomaterial as described above and further a step of implanting the biomaterial in a vertebrate. For example a mammal, including a human.

In general, the implantation may be carried out for purposes of replacing and/or reconstructing a bone in the body, for example as a consequence of an injury or disease. It may also be used to replace and/or reconstruct periodontal defects and periodontal regeneration. The implantable biomaterial according to the invention may also be used to bone augmentation in general.

For example such grafts would be indicated in orthopaedic applications and in particular when the graft is put under load, ie in particular: spinal surgery (cervical fusion, replacement of lumbar discs, etc), reconstruction of the base of the cotyle, arthroplastic surgery, osteotomy, pseudoarthrosis, arthrodesis, and the like.

The implantable biomaterial according to the invention is a natural anorganic bone implant material that has almost the same composition as a human bone, and it is used as a temporary scaffold where bone cells of the patient can grow. Since this implant has the right composition and 3-dimensional architecture of a bone, the body would eventually replace the scaffold with natural bone tissue, and naturally remodel the implant. There will also be no mechanical mismatch between this implant and that of existing tissues as they are made of the same material, and have identical structures.

According to a further aspect, the invention may also comprise a further step of cultivating the implantable biomaterial in vitro prior to implantation. Further, the method may comprise a step of seeding the implantable biomaterial with the patient's own cells prior to implantation.

In particular, the composition of the implantable biomaterial obtained or obtainable according to any embodiment of the method of the invention has a Ca/P ratio of 1.64.

In particular, the method of the invention does not comprise a step of treating the bone tissue with a fluid in supercritical state.

According to a further aspect, the invention provides an implantable biomaterial prepared according to any embodiment of the method of the invention. In particular, the implantable biomaterial has a Ca/P ratio of about 1.64.

The implantable biomaterial may be cultivated in vitro. For example, the implantable biomaterial may be seeded with the patient's own cells prior to implantation.

Accordingly, the invention also provides an in vitro cell culture comprising the implantable material prepared according to any embodiment of the method of the invention. The in vitro cell culture may comprise the implantable biomaterial seeded with the patient's own cells.

The implantable biomaterial according to any embodiment of the invention solves the problems existing in the prior art associated with processing the bone tissue, and provides an implantable bone tissue with high osteoinductivity, osteoconductivity, biocompatibility, and comparable mechanical strength to natural bone. Most importantly, it is a very low cost and simple process to treat animal bone in the manner proposed.

Further advantages of the invention are that all the processing involves natural treatments and mild solvents without toxic chemicals, proteases, and without requiring treating the bone tissue with a fluid in a supercritical state. The repeated boiling process and/or ultrasonic treatment are excellent for disrupting the bone marrow and ECM without changing the physical and chemical properties of the bone scaffold. According to the invention, the ECM and bone marrow cells will be removed from the bone tissue and only the bone scaffold will remain. Such a material has almost the same properties as a natural bone. It is therefore highly osteoconductive, and safe for implantation without the risk of viral infection or immunological rejection.

Having now generally described the invention, the same will be more readily understood through reference to the following examples which are provided by way of illustration, and are not intended to be limiting of the present invention.

### EXAMPLES

### Example 1

Natural bones of essentially all vertebrates have a basic structure of hydroxyapatite (HA), formulated as [Ca₁₀(PO₄)₆(OH)₂]. However, crystals of HA found in biological tissues such as bone, enamel, dentin and other calcified tissues contain other atoms and ions such as acid phosphate groups (HPO₄²⁻), carbonate ions (CO₃²⁻), magnesium (Mg), fluorine (F) (LeGeros R Z., Crystal Growth Charact. 1981; 4:1-45; Rey C, et al., Calcif. Tissue Int. 1991; 49:251-258). Bone crystals either do not contain hydroxyl group or contain very few such groups, referred to as carbonate apatite rather than HA (Bonar L C, et al., J Bone Miner Res. 1991; 1167-1176). The groups of carbonate and phosphate in bone crystals are relatively unstable and very active, which result in an important role in the bone formation, mineralization and dissolution (LeGerps R Z. Tung M S., Caries Res. 1983; 17:419-429).

The majoring of synthetic HA preparation for bone substitutes are of synthetic origin and distinct structurally and chemically from the biological calcium phosphate crystals in bones. Pure hydroxyapatite is essentially nondegradable and the resorption rate of HA is only 5-15% per year (Fleming J E Jr, et al., Orhop Clin North Am. 2000; 31: 357-374). Calcium phosphate ceramics are more brittle and have less tensile strength than bones (Jarcho M., Clin Orthop. 1981; 157:259-278; Truumees E, et al., Univ of Pennsylvania Orthopaedic J 1999; 12:77-88). These synthetic calcium phosphate crystals are not only chemically and structurally distinct from the apatite crystals of bones, but in some cases, they contain varying amounts of amorphous calcium phosphate, which are not crystalline at all (Fleming J E Jr, et al., Orhop Clin North Am. 2000; 31: 357-374; Truumees E, Herkowitz H N., Univ of Pennsylvania Orthopaedic J 1999; 12:77-88; Bohner M., Injury. 2000: 31: SD37-SD47). In some cases, synthetic calcium phosphate also contains calcium salts such as calcium oxides. These additional calcium salts have been the subject of an extensively long term study to investigate the effect on bone mineralization, dissolution and further biological functions related to osteoblasts.

Natural bones contain approximately one third of organic constituents and the major component of them is collagen fibrils (Cohen-Solal L, et al., Proc Natl Acad Sci USA. 1979; 76:4327-4330). The covalently phosphorous bond of collagen or non-collagenous proteins with apatite in bone makes it difficult to isolate natural animal bones from organic matrix without producing significant changes in the chemistry and structure of crystals (Sakae T, et al., J Dent Res. 1988; 67:1229-1234). In clinical trials using anorganic bones, good osteoconductivity, osteointegration and good defect resolution in various situations has been demonstrated (Richardson C R, et al., J Clin Periodont. 1999; 26:421-428; Young C, et al., Int J Oral Maxillofac Impl 1999; 14:72-76; Lorenzoni M, et al., Int J Oral Maxillofac Impl. 1998; 13:639-646). However, there are certain contra-indicated in orthopedic surgeries (Sciadini M F, et al., J Orhto Res. 1997; 15:844-857; Jensen S S, et al., Int J Oral Maxillofac Impl. 1996; 11: 55-66; Raspanti M, et al., Biomater 1994; 15:433-437), such as a reduced resistance to mechanical stress, a process of recrystalization, and a newly formed HA salts.

Commercial and experimental bone graft materials can show a variety of compositions and properties, many of which are very different from those of natural bones (Boyne P J. Comparison of Bio-Oss and other implant materials in maintenance of the alveolar ridge of the mandibule in man. In: Huggler A H, Kuner E H, editors. Heft'e zur unfallheinde 216. Berlin: Springer' 1991. p11; Lorenzoni M,et al., Int J Oral Maxillofac Impl 1998; 13:639-646; McAllister B S, et al., Int J Periodont Restor Dent 1998; 18:227-239). Inorganic calcium derivatives are frequently used such as phosphate ceramics, tricalcium phosphate, calcium phosphate cements, nanoparticle HA and calcium sulfate. The physicochemical properties of these materials had been investigated depending on their interfaces with host bone and described as bioinert or bioactive. Although bioceramics provide a scaffold for the ingrowths of bone from the adjacent host bone, they have no inherent bone forming ability and also provide limited mechanical strength and hindered by the variable resorption (Jarcho M., Clin Orthop. 1981; 157:259-278). Alternatively, natural bone minerals have been clinically used in various situations to facilitate the growth of new bones into osseous defects (Richardson C R, et al., J Clin Periodont. 1999; 26:421-428; Young C, et al., Int J Oral Maxillofac Impl 1999; 14:72-76; Lorenzoni M, et al., Int J Oral Maxillofac Impl. 1998; 13:639-646). The method according to the invention represents a procedure for purifying natural bone free of organic matrix without disrupting the natural crystalline structure. The characterization is emphasized in physico-chemical properties with the aid of XRD analysis, SEM-EDX and mechanical and elasticity test.

Materials and Methods

Scanning Electron Microscopy Investigations

The anorganic cortical and spongiosa bones were dissected 1 cm×1 cm×0.5 cm before being autoclaved as indicated in the preparation of an anorganic bone. Scanning electron microscopic (SEM) investigations were carried out on the basic prepared anorganic porcine bone with dense and sponge and the surface of crystalline structure from the bone. The studies were carried out after sputtering the samples with gold.

The crystalline structures from the dense and spongiosa anorganic bones were observed and constituents of each crystalline were analyzed by SEM-EDX (Hitachi 4200).

Determination of Physical Properties

Mechanical Strength and Elastic Modulus Testing Method

Cortical bones from treated porcine femora were cut into 5 mm×3 mm×6 mm dimension typically. The bones were loaded along the longitudinal axis on Instron 3345 (Instron Corporation, Canton, Mass.). Crosshead speed of the tester was 1 mm/min. All data was collected from dry bone with an environmental humidity of 50% at 23° C.

Porosity Test Method

Sponge bone (10 mm×10 mm×10 mm) was put into the penetrometer of a mercury porosimeter (Autopore III 9420 from Micromeritics) directly. The sample was analyzed from 0 to 60,000 psi.

Isolation of Natural Bone Free of Organic Matrix

A method was developed to remove organic matrix and yields anorganic calcium phosphate scaffolds from the dense and spongiosa bones. The isolation of natural bone free from organic matrix (the implantable biomaterial) was prepared according to the stages as follows.

Anorganic Porcine Bone (APB) Preparation

The anorganic porcine bone (APB) was obtained by removing the epiphyseal and diahyseal region of fresh swine femora.

Preparation of Porcine Long Bones

A fresh porcine long bone chopped into several pieces was obtained from the slaughterhouse. Pieces of approximately 1-3 cm³ were cut for further treatment. The pieces of bone were cleaned with tap water thoroughly and boiled in the water for 2 hrs with frequent changing of the water approximately every 30 minutes to remove the slurry. The material was cleaned with water and removed from the disassociated cartilages. After that it was cleaned with deionized water and oven-dried for 2 hrs at 210° C., occasionally changing the position to prevent overheating in certain areas. At this point, the disaggregated organic matrix was partially removed by such treatment. Under such treatment, bone marrow was completely removed.

Dissociation of Bone Matrix Constituents

After drying, the material was autoclaved at 121° C. for 15 minutes to have minimized sterilization. The pieces of bone were immersed in 70% ethanol for further removing the oil and loosening organic matrix attachment. They were then ultrasoniced for 30 min at 37° C. for further removal of the aggregated organic matrix (mainly collagen) with sterilization effect contributed from 70% alcohol. This step lasted for about 4 hrs until the solution had no turbidity. The material was replaced with fresh 70% alcohol for each 30-min period. The ultrasound procedure was performed for a period of 2 to 3 minutes at 270 watts, 63 KHz, peak output frequency. Finally, the treated material was autoclaved in 70% alcohol at 121° C. for 15 min to have maximal sterilization.

The architecture of trabecular and cortical bones was kept intact within a smaller size of dimension. The structures of the treated bones were shaped and sized between 3 cm and 0.5 cm in this experimental study.

Characterization of Anorganic Xenografts

Crystalline Structure by XRD

To determine the composition of different minerals of anorganic porcine bone after several stages of treatment, X-ray diffraction (XRD) was used for the fingerprint characterization of the crystal structure and to determine its structure. XRD of small pieces of such anorganic porcine bone showed no significant changes in the crystal components (FIG. 3).

The XRD patterns of the derived anorganic bone and the synthetic hydroxyapatite are nearly identical. The anorganic bone showed typical diffraction peaks at 211 and 002 of typical calcium phosphate compared to the synthetic HA. Although the anorganic bone is similar to synthetic HA, the small crystals from the anorganic bone are represented in the XRD analysis by broad interference lines, resulting in a very broad spectrum. The small interference lines represent the crystals of the synthetic HA and showed a characteristically narrow spectrum.

The crystal structure in the synthetic HA is, therefore, more vigorous than those in the anorganic bone, which make the anorganic bone more adaptive to the process of bone formation.

Chemical Composition Determined by SEM-EDX

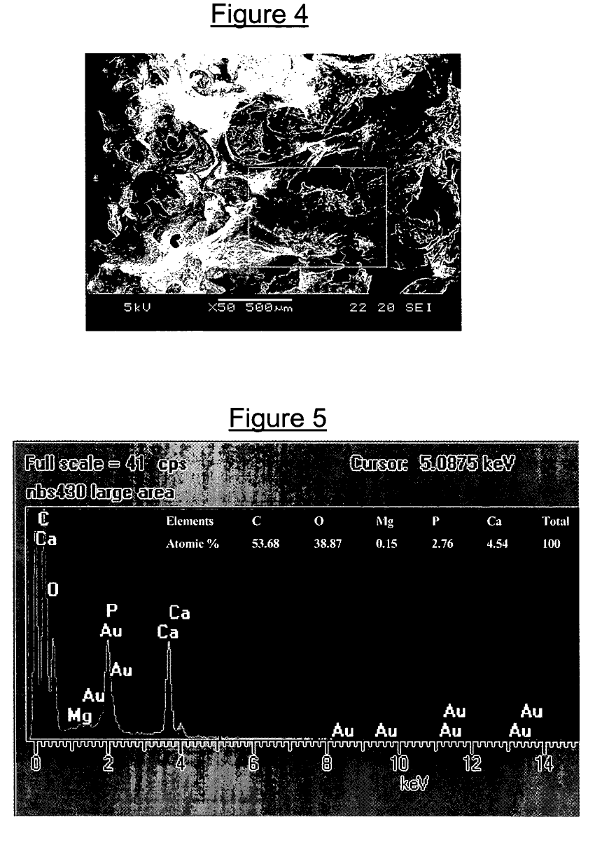

The overall composition of anorganic porcine bone had been analyzed by scanning electron microscopy-energy dispersive x-ray spectroscopy (SEM-EDX, Hitachi 4200) (FIGS. 4 and 5). The treated materials retained the natural mineral content of the bone, which have a typical Ca/P ratio of 1.64, slightly lower than the human bone, which is around 1.71 (Ref: LeGeros, R. Z., Apatites in biological system. Prog. Crystal Growth Charact., 4, 1-45, 1981). In addition to the standard Ca/P ratio indicating the composition of HA, there were other minerals such as Mg found in the anorganic porcine bone. This may possibly be one of the reasons that make the HA have a lower ratio compared to the human bone.

Physical Properties of Anorganic Porcine Bone

Porosity

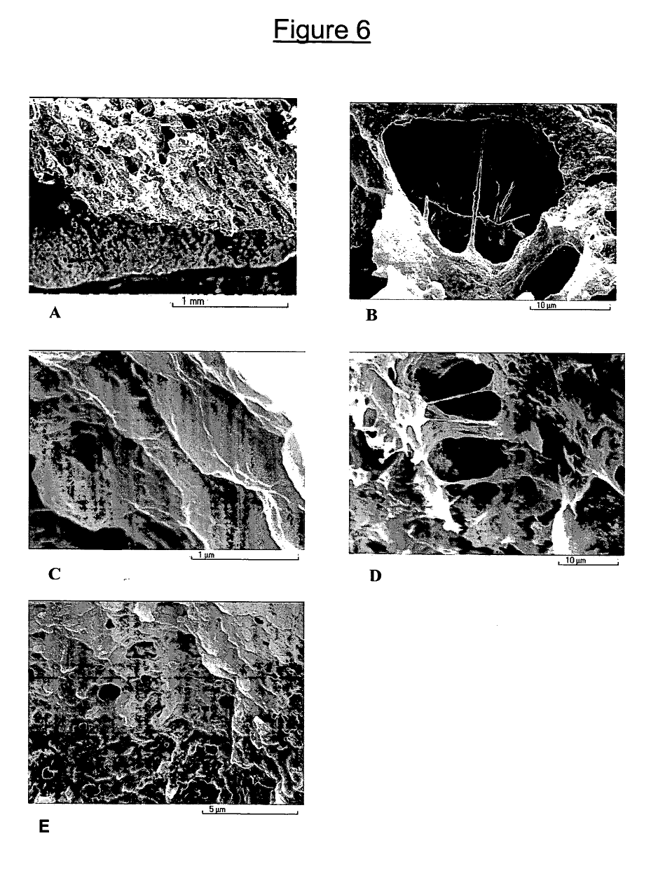

The typical structures of dense and spongiosa bones are shown in FIGS. 6A-E analyzed by SEM. The results demonstrated the side interconnective pore system of natural bone minerals. In general, the natural bone mineral of porcine bone consists of the macropores (FIG. 6A), micropores in dense (FIG. 6B) and sponge (FIG. 6D), and intercrystalline spaces in dense (FIG. 6C) and in sponge (FIG. 6E). The crystal sizes were directly measured the scaffold cross section from SEM-EDX images (FIG. 6C). Through measuring the largest axis of the assessed pore, the size of microcrystals was approximately around 100 nm. The systems resulted in an overall high porosity of 65% and inner surface of the natural bone. The high porosity and inner surface will greatly enhance the penetration of host bone repair into the inner part of the graft materials.

Physical Properties

The measurement showed that the compressive strength of an anorganic cortical bone was 40.9 MPa, which is in the same range as compared to that of the human cortical bone, which is 40 MPa. The synthetic HA showed high compressive strength that represents a high stiffness and a high density for synthetic materials in comparison to the surrounding host recipient bone.

The modulus of elasticity for anorganic cortical bone was 1.1 Gpa in maximal strength, and the average strength was around 621.4 MPa. On the other hand, synthetic HA showed a higher modulus of elasticity (34-100 Gpa) and resulted in low flexibility.

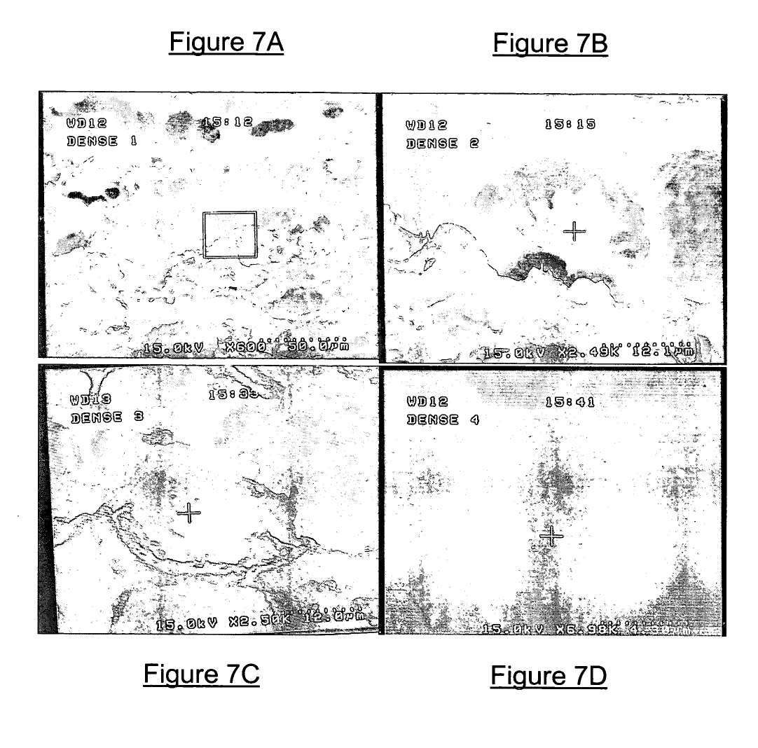

TABLE 1
(FIG. 7A)

Elements

| | Ca | O | Mg | Al | C | P |
|---|---|---|---|---|---|---|
| Atomic % | 3.61 | 47.18 | 0.16 | 0.16 | 44.81 | 4.08 |

TABLE 2
(FIG. 7B)

Elements

| | Ca | O | Mg | Al | C | P |
|---|---|---|---|---|---|---|
| Atomic % | 0.43 | 53.43 | 0.001 | 0.002 | 46.14 | — |

TABLE 3
(FIG. 7C)

Elements

| | Ca | O | Mg | Al | C | P |
|---|---|---|---|---|---|---|
| Atomic % | 6.83 | 68.71 | — | 0.36 | 17.09 | 7.01 |

TABLE 4
(FIG. 7D)

| | Elements | | | | | | |
|---|---|---|---|---|---|---|---|
| | Ca | O | Mg | Al | C | P | Na |
| Atomic % | 1.45 | 30.63 | 0.001 | 0.81 | 63.28 | 3.77 | 0.001 |

Distribution of Non-Homogenous Constituents of Crystallines in Anorganic Dense and Spongiosa Bones
Chemical Composition of Individual Crystallines in Anorganic Dense Bones

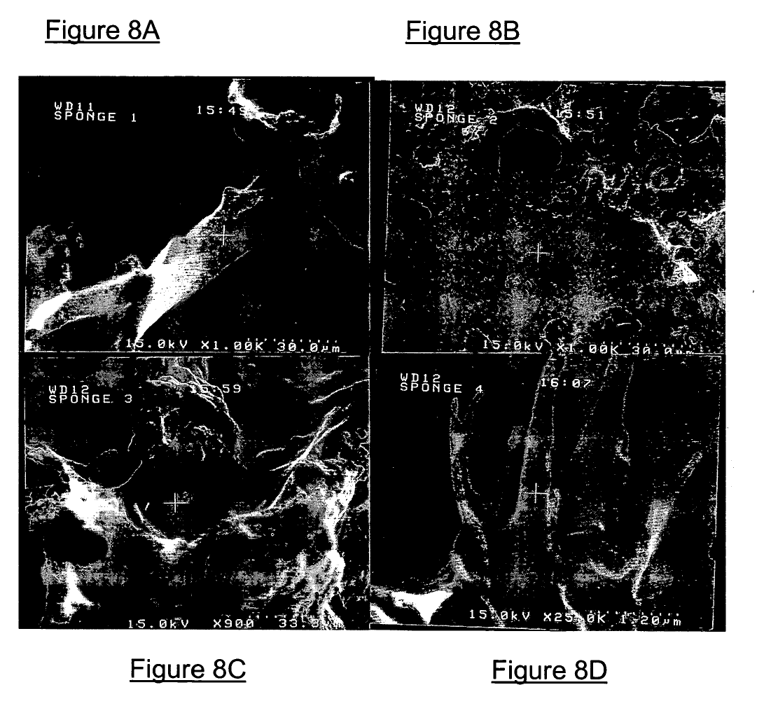

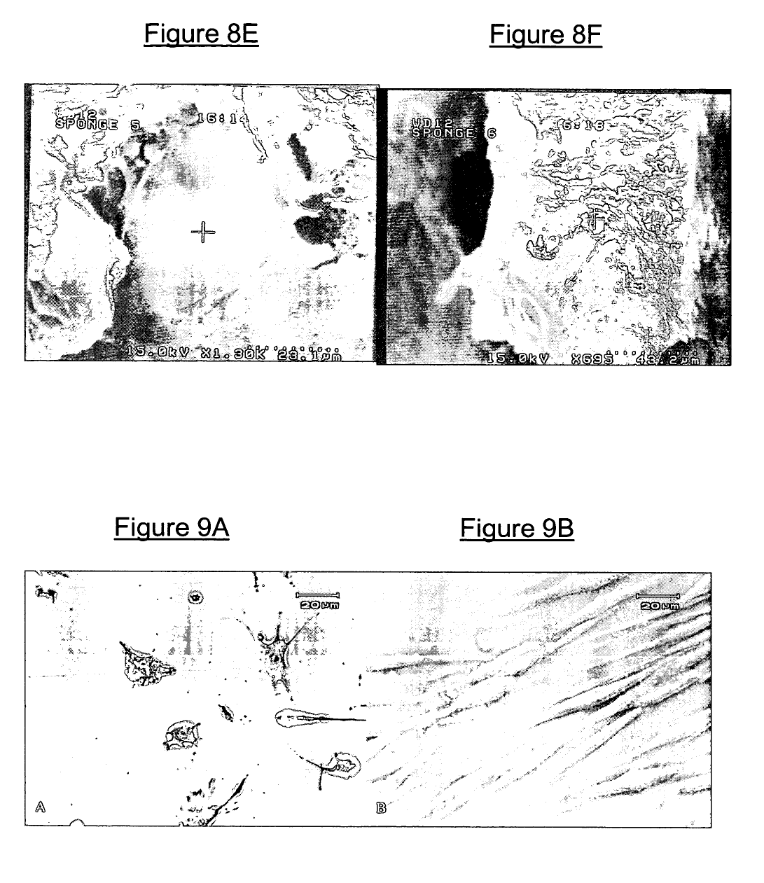

Although there are typical structures from natural bones as indicated by FIGS. 6, 7 and 8, it is possible to differentiate several different kinds of unique crystallines from dense and spongiosa bones. The chemical composition of a graft material influences the rate and extent to which it is incorporated into the host tissue and the subsequent physical characteristics of the graft site. In considering the bone remodeling, the compositions of graft materials also influence bone dissolution, mineralization as well as formation. To differentiate the chemical compositions of each individual crystalline, four individual crystalline structures from the dense bone and six from the spongiosa bone were selected for SEM-EDX analysis (FIGS. 7 and 8).

The morphology and components are shown in the micrographs presented in FIGS. 7(A, B, C and D) for the dense bone and FIGS. 8(A, B, C, D, E and F), for the spongiosa bone. The characteristics and components of each crystalline in dense bone suggested the existence of the element aluminum, Al, in all crystallines, just with different atomic % (Table 1-4, indicated as dense 1 to dense 4 respectively). The element sodium, Na, only appeared in the crystalline of dense 4 (FIG. 7D, Table 4). The crystalline constituents of dense 2 (FIG. 7B, Table 2) and dense 3 (FIG. 7C, Table 3) did not have the typical element of phosphate (P) or magnesium (Mg), individually, compared to the overall crystal structure of the anorganic porcine bone in FIGS. 4 and 5. The ratio of Ca/P of each individual crystalline was all below 1. The atomic % of aluminum (Al) had a very variable range within these four crystalline structures. The smallest % of Al is 0.02% in dense 2 (FIG. 7B, Table 2) and could be 400× increments in dense 4 (FIG. 7D, Table 4).

Compared to the dense bone, the six crystalline structures from the spongiosa bone showed similar constituents with different element combination (FIG. 8A-F, sponges 1 to 6 respectively). Four of the six crystalline structures were deficient in the element Al, and the atomic % of Al in the spongiosa 1 and 2 was quite equivalent. The ratio of Ca/P of all crystalline structures was below 1, similar to the crystalline structures from the dense bone. It was also observed that there was a higher atomic percentage of sodium in the spongiosa bone than in the dense bone.

Comparison of constituents from each crystalline that we have analyzed is listed in Tables 5-10.

TABLE 5
(FIG. 8A)

Elements

| | Ca | O | Mg | Al | C | P |
|---|---|---|---|---|---|---|
| Atomic % | 0.27 | 54.00 | — | 0.20 | 45.55 | — |

TABLE 6
(FIG. 8B)

Elements

| | Ca | O | Mg | Al | C | P |
|---|---|---|---|---|---|---|
| Atomic % | 4.73 | 75.11 | — | 0.17 | 15.15 | 4.84 |

TABLE 7
(FIG. 8C)

Elements

| | Ca | O | Mg | Al | C | P |
|---|---|---|---|---|---|---|
| Atomic % | 0.04 | 35.47 | — | — | 64.49 | — |

TABLE 8
(FIG. 8D)

| | Elements | | | | | | |
|---|---|---|---|---|---|---|---|
| | Ca | O | Mg | Al | C | P | Na |
| Atomic % | 1.27 | 92.25 | 0.13 | — | — | 5.85 | 0.50 |

TABLE 9
(FIG. 8E)

Elements

| | Ca | O | Mg | Al | C | P |
|---|---|---|---|---|---|---|
| Atomic % | 1.44 | 72.88 | — | — | 22.33 | 3.35 |

TABLE 10
(FIG. 8F)

| | Elements | | | | | | |
|---|---|---|---|---|---|---|---|
| | Ca | O | Mg | Al | C | P | Na |
| Atomic % | 4.74 | 44.42 | 0.10 | — | 44.59 | 6.04 | 0.11 |

The results from the SEM-EDX analysis demonstrated that the porcine natural bone has overall similar components as the natural bone as the Ca/P, which is 1.64. Interestingly, there is non-homogenous distribution of basic elements like Ca, P, C, O, and Mg in most of the area based on the crystalline structures. Extra elements such as Al and Na, if not all, are commonly added in different crystalline structures, as some of crystallines may not consist of some basic elements like P, C, and/or Mg.

Discussion

In this experiment, the data clearly demonstrates that the method of the invention for preparing an anorganic porcine bone (implantable biomaterial) maintains the intact architecture, the crystal structure and the chemical components of dense and spongiosa bone analyzed by XRD and SEM-EDX. The Ca/P ratio of APB (1.64) is slightly lower than the hydroxyapatite (1.67). Biological apatites differ from the pure HA in composition, crystal size and morphology as determined by age, other minor elements such as Mg, carbonate, Na, Cl, phosphate, etc. and trace elements such as strontium, Sr; lead, Pb; chromium, Cr; zinc, Zn; Nikel, Ni.; etc. (Featherstone, J D B, et al., Calcif. Tissue Int. 1983; 35:169-171; McConnel, D., Biochim. Biophys. Acta. 1980; 32:169-174; Brown W E, Chow L C., Ann. Res. Mater. Sci. 1976; 6:213-226; Arends J, Davidson C., Calcif. Tiss. Res. 1975; 18:65-79; LeGeros R Z., Arch. Oral Biol. 1974; 20:63-71; LeGeros R Z, Bonel G., Calc. Tiss. Res. 1978; 26:111-116). The formulated Ca₁₀(PO₄)₆(OH)₂ for pure HA can be replaced by (Ca, Na, Mg, K)₁₀(PO₄, CO₃, HPO₄)₆(OH, Cl, F)₂ for biological apatite. The low Ca/P of APB could be explained by other minor substitutes with Ca, such as Mg, Na, and Al in our case. The percentage of element C is the critical factor in contributing to the ratio of Ca/P, which carbonated apatite is a dominant structure of within all crystalline structures the authors have analyzed. It has been reported that the carbonate concentration is also highest in dentin and bone (LeGeros R Z. Incorporation of magnesium in synthetic and biological apatites: a preliminary report. In: Tooth Enamel IV, Tearnhead R W, Suga S; Eds. Elsevier: Amsterdam: 32-36, 1984).

The XRD pattern of ABP suggests that the whole structure remains well intact in terms of carbonate apatite with minor Mg substitutes. The ABP also well preserves physical properties in terms of mechanical strength, elasticity and crystalline structures including macropores, micropores and intercrystalline spaces.

During the process of preparing the anorganic porcine bone, critical considerations taken into account included mild solvent treatment, where only 70% ethanol was applied; maintaining the temperature below 210° C., in order to avoid of high temperature which may have induced changes in the crystal structure. When the crystal structures of the bone are heated to temperatures above 400° C., recrystallization occurs and the crystals tend to grow, thus causing the structure and chemical components to change (Raspanti M, et al., Biomater 1994; 15:433-437).

As a result, the anorganic porcine bone retains the natural mineral content of the bone, which preserves the complex composition compared to synthetic hydroxyapatite. The form of anorganic porcine bone has a high surface/volume ratio as interpreted in FIG. 6 and offers a large surface for interaction, a correct three dimensional structure possibly required for directing the cell proliferation, differentiation as well as apoptosis.

A synthetic material produced in the laboratory has not replicated the complex surface of natural porcine bone as shown in FIG. 6. Each unique non-homogenous crystalline structure composing the structure of spongiosa and dense bone further builds up the complexity network of scaffold for directing cell proliferation, differentiation and bone formation. The natural bone does not only provide the source for building up the new bone, it also provides the complicated 3D structure as well as a network of combination of biomaterials. Such a complicated system for directing bone remodeling is not easy to be reproduced by any synthetic materials.

### Example 2

Dissolution of Organic Bone

An anorganic bone is an ideal biomaterial for in vitro analysis of bone dissolution/precipitation. The anorganic porcine bone, free of organic matrix, maintains the physicochemical properties and crystalline structures as the natural bone (as shown in Example 1). However, little is known about the deposition of natural bones due to certain experimental difficulties. Thus, the aim of this Example is to investigate the dissolution/precipitation of crystalline structures from the anorganic porcine bone encountered with or without osteoblasts cells, and to emphasize various observations related to bone mineralization under the physiological conditions. The deposits contain several elements including Ca and/or P. The ratios of Ca/P are less than 1 in all deposits as the dissolution rate of each element depends on the composition of each corresponding site. The present inventors have reported that non-homogenous distribution of essential/trace elements along the cortical and spongy bones. The commonly proposed mechanism underlying the phenomenon of the bioactivity of biological anorganic apatite involves the dissolution of calcium, phosphate, silicon and trace/essential elements from the apatites.

Accordingly, the Ca/P ratios obtained as result of the dissolution/precipitation Example 2 are therefore different from the Ca/P ratio of 1.64 of the composition of the organic bone obtained in Example 1.

Materials and Methods

Anorganic Porcine Bone

The anorganic porcine bone (APB) (also indicated as implantable biomaterial) was obtained as in Example 1. The treated samples were autoclaved (121° C. for 15 min) before testing. Under such treatment, the trabecular and cortical bone architectures were kept intact. A commercially synthetic calcium phosphate scaffold, termed BDS (BD Biosciencez, Bedford, USA), was used as a control.

Cell Culture Procedure

Osteoblastic cells, MC3T3-E1, were cultivated in α-minimal essential medium (MEM), supplemented with 10% fetal bovine serum (FBS), 50 Uml⁻¹ streptomycin and 50 μgml⁻¹ penicillin (Gibco). Myoblastic cells, C2C12, were cultivated in Dulbecco's modified Eagle's medium containing 4 mM glutamine, 1.5 g/L sodium bicarbonate and 4.5 g/L glucose, 10% of FBS and 50 Uml⁻¹ streptomycin and 50 μgml⁻¹ penicillin (Gibco). Observations of cell morphology were performed using an inverted phase contrast light microscope (Olympus, CKX41).

Preparation of Scaffolds

The anorganic porcine bone (APB) was prepared as a scaffold of 0.5 cm³. A commercially synthetic calcium phosphate (BDS; BD Biosciences) 5 mm in diameter was also used in the experiment.

In order to study the deposition of minerals, the scaffolds of APB and BDS were pre-incubated in culture media for 1 day at 37° C. The purpose of pre-incubation of scaffolds with media is to completely hydrate the scaffolds.

In Vitro Mineralization

The 0.5 cm³ scaffold was placed in a 24-well plate. Approximately 5×106 cells in 1 ml of culture media were seeded onto the scaffolds and incubated at 37° C. overnight. The media was changed every 4 days until the end of the experiment.

Analyses

Alkaline Phosphatase Assay

Akaline phosphatase acitivity was measured using AP Assay Kit (Sigma Diagnostics, Switerland). In brief, late passage cells in 12-well plates with either no APB or treated with APB of 1 mm³ for 1-, 2- and 3-days, individually. At the time indicated, the cells were rinsed twice with ice cold PBS (pH 7.4) and resuspended in ice cold PBS, then centrifuged at 600× g for 2 min. The pellet was resuspended in 6 μl of ice cold PBS. The sample (2 μL) was added in the 10 μL of solubilized solution, pH 10.5 (5 mM Tris, 100 mM glycine, and 0.1% TritonX-100) and lysed for 5 min. at room temperature. The solubilized samples were measured at OD₄₅₀. Alkaline phosphase was expressed as nmol p-nitrophenol phosphate per μg protein/min. total proteins to get IU/L/μg). Total protein was measured by the Bio-Rad protein assay kit II (BioRad, Glattbrugg, Switzerland) using bovine serum albumin as a standard.

Transmission Electron Microscopy-Energy Dispersive X-Ray Spectroscopy

The media was analyzed on days 7 and 14. Mineral analysis was based on transmission electron microscopy-energy dispersive x-ray spectroscopy (Jeol 3010; TEM-EDX).

After 1 week and 2 weeks of seeding, the media from the culture was collected, respectively. The collected media was centrifuged at 3,000 rpm for 10 min to remove the cell debris. Then 100μl of the media was absorbed on the carbon coated TEM copper grid at room temperature. The excess liquid was removed with a piece of filter paper and the grid was air-dried.

Scanning Electron Microscopy-Energy Dispersive X-Ray Spectroscopy

The media alone or the media containing osteoblasts with APB on the coverslips for 2 weeks were analyzed. The media was removed and the precipitates on the coverslips were air-dried, then coated with Au. The samples were analyzed by using a scanning electron microscopy-energy dispersive x-ray spectroscopy (SEM-EDX, Hitachi 4200).

X-Ray Diffraction

X-ray diffraction (XRD) was recorded with a Shimadzu diffractometer (λCuKα, 30 mA, 40 kV) equipped with a graphite back-monochromato, using the stepscan procedure with a 0.02 sampling pitch and a 2°/min scan rate. The media was individually absorbed in the sponge with the size of 2 mm³ at room temperature and dried in air.

Results

Culture Morphology

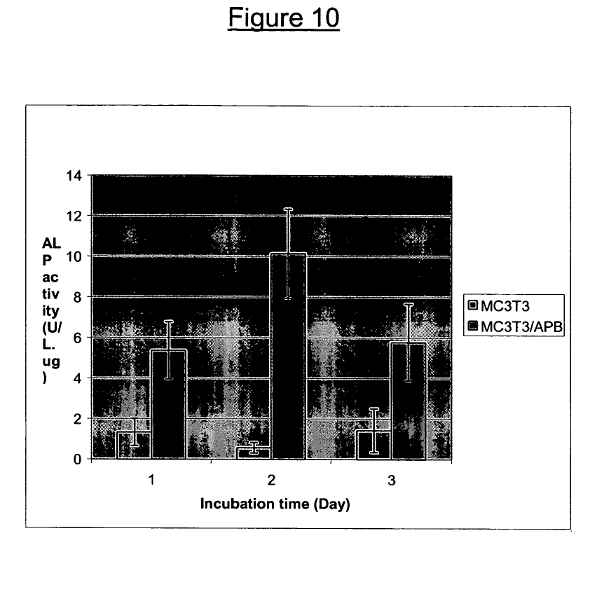

The anorganic porcine bone (APB) of 5 mm³ was seeded with the osteoblasts, MC3T3-E1. After 14 days incubation, the cell morphology was observed under light microscopy. The cells encountered with APB were observed to have a very elongated shape with smooth edges (FIG. 9B). The length of APB-directed cells could be extended to 200 μm as the normal cells had a very irregular shape with the average size of 30 μm (FIG. 9A). Such elongated cells were only observed when the cells were incubated with APB only without close contact (data not shown). In addition, an increase in the number of incubation days of cells with APB correlated with elevated levels of alkaline phosphatase activity (FIG. 10), which is a marker for osteoblasts differentiation. There was no obvious change of ALP when the media was collected from the osteoblastic cells alone. Taken together, these findings indicate that the APB provided a suitable environment for in vitro analyses of osteoblasts in the process of bone formation.

The Formation of Crystalline Structures During APB Incubated with Osteoblasts Cells

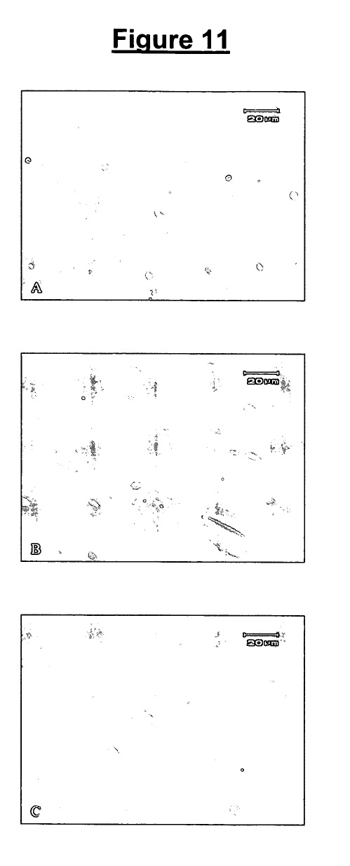

During the incubation of osteoblasts and APB, some crystalline structures were mineralized and precipitated in the dish (FIG. 11A-C). The shell-like structures with a very organized order either with pore-like structures (FIG. 11A), straight lines (FIG. 11B) or both (FIG. 11C) on the surface. These crystalline structures will form following a straight route from a smaller size and extend to a bigger size.

Analysis of Components Deposited from the Media Containing Cells with APB

The dissolution of anorganic porcine bone and the formation of crystalline structure provide great information for cell differentiation as well as bone formation.

The experiment of Example 1 showed that the dense and spongy bones generally contained P, O, C, Ca and Mg by SEM-EDX. Non-homogenous distribution of P, O, C, Ca, Al, Na, and Mg in each crystalline was observed within dense and spongy APB. There were different molar ratios of Ca/P of each individual crystalline structure depending on the loci. The 3-dimensional (3-D) structure is complicated not only by the 3-D architecture, but also the component of each crystalline structure. To examine the deposits from the media containing cells with APB, osteoblastic cells were plated in a concentrated manner (6×10⁵ cells/ml) into the APB with a size of 0.5 mm³. After one week of incubation, component analysis from several distinguished crystalline structures under such conditioned media were examined by TEM-EDX. The same media without APB and cells was used as a control. Based on the media used, the component of each crystalline structure deposited from the conditioned media could either be from the media itself or the dissolution of APB or both. The basic components from the control media for deposition were Na, K, P, S, Ca, C and Mg. Several deposits from the conditioned media contained most of the basic components Table 11).

TABLE 11
Elements in different solids in solution containing NBS and 3T3
cells for 1 week, and contents of solid from medium is listed as a comparison

| Solid 1 | Solid 2 | Solid 3 | Solid 4 | Solid 5 | Solid 6 | Medium |
|---|---|---|---|---|---|---|
| Na | Na | Na | Na | Na | Na | Na |
| K | K | K | K | K | K | K |
| Cl | Cl | Cl | Cl | Cl | Cl | Cl |
| P | P | P | P | P | P | P |
| S | S | S | S | S | S | S |
| Ca | Ca | Ca | Ca | Ca | Ca | Ca |
| | C | | | | | |
| | Cr | | | | | |
| | | Mg | | Mg | Mg | Mg |
| | | | | Si | Si | |
| Ca/P = 0.5 | Ca/P = 0.94 | Ca/P = 1.07 | Ca/P = 0.64 | Ca/P = 0.77 | Ca/P = 2.7 | Ca/P = 0.92 |

Generally, the deposits from the conditioned media had a very high atomic % of element Cl, which were 42%, 57% and 34%, and a very low atomic % of Ca. In contrast, there was a very low atomic percentage of Cl in the control, which was 1.5%, 1.2% and 2.6%. In addition to other similar components of each crystalline structure with the distinguished different molar ratios of certain elements, some extra elements, including Cr, Si, and Co could be dissolved from the APB and mineralized with some of the deposits. Two of six crystalline structures contained the element Si.

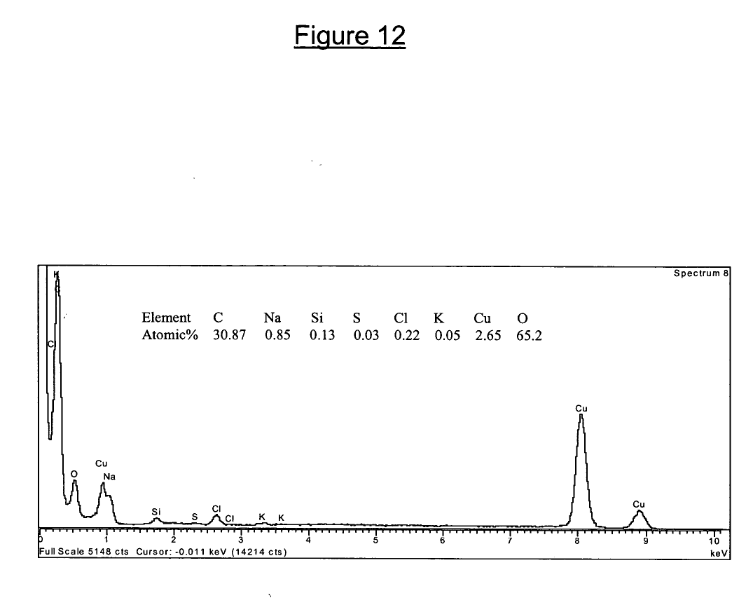

To prevent the contamination of element Si from either the glass-based coverslip or the containers during the preparation of APB, the silicon-free APB was prepared with no glass contact during the preparation of the microscopic examination. Components of the crystalline structure from such silicon-free APB with osteoblasts contained the element Si as shown in FIG. 12. The control media with or without the glass-based coverslip showed no silicon in each deposit examined. The deposits from the same cells with a synthetic calcium phosphate scaffold (BDS) were also examined. No silicon or trace elements were detected under the same conditions.

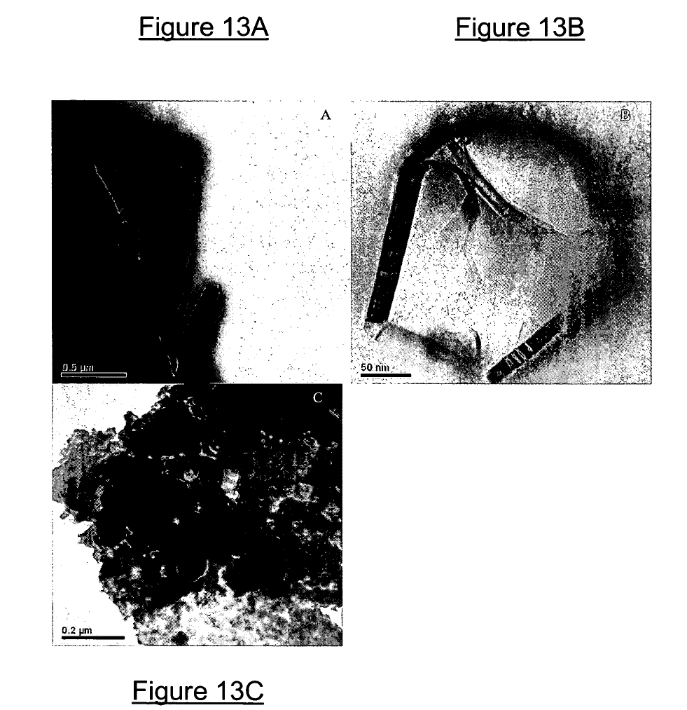

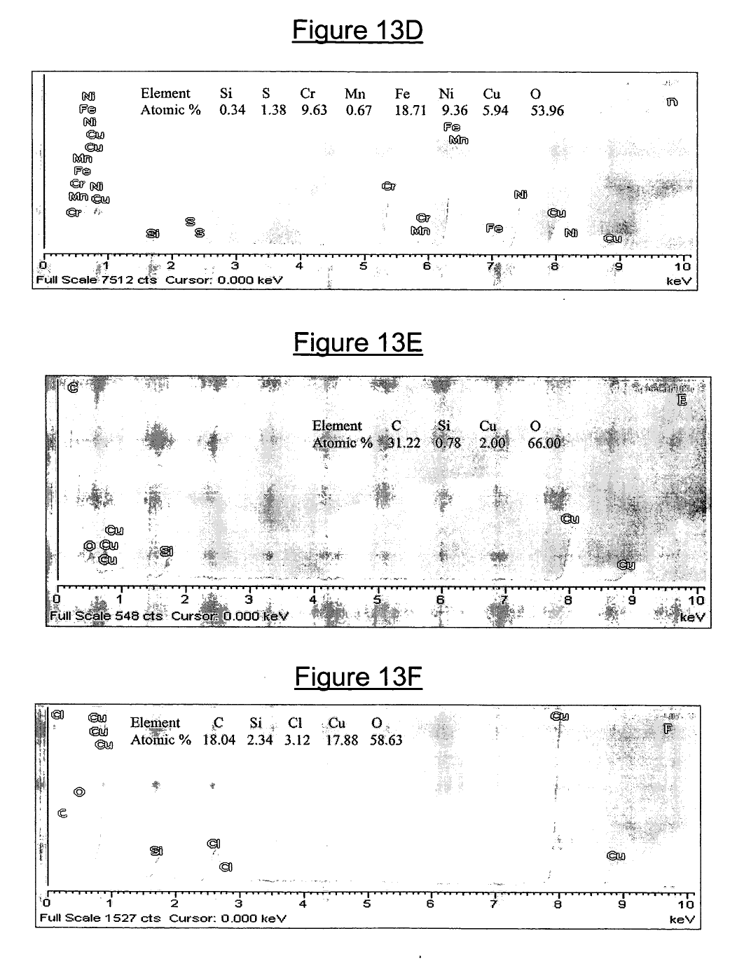

To evaluate the time-dependent mineralization, osteoblasts cells encountered with APB for 2 weeks were examined. The component profiles from the 2-week deposits were significantly different from those of 1 week. The basic components were S, C and Si. The element Si was deposited in each solid examined. A variety of trace elements were also found, such as Cr, Mn, Fe, and Ni (FIG. 13B). The crystalline structure with trace elements showed a brunch-like morphology (FIG. 13A), and a well-organized needle structure had a simple Si-containing carbonate (FIGS. 13C and D) or a Cl, Si-contaning carbonate (FIGS. 13E and F), respectively.

Osteoblast-Independent Mineralization

The deposition/mineralization of trace elements and Si have raised the question whether specific cells regulated the process. To answer the question, two culture conditions were prepared. One was the APB incubated in the normal culture conditions with no cells added. The other was the APB incubated with non-osteoblasts, such as myoblast cells, C2C12.

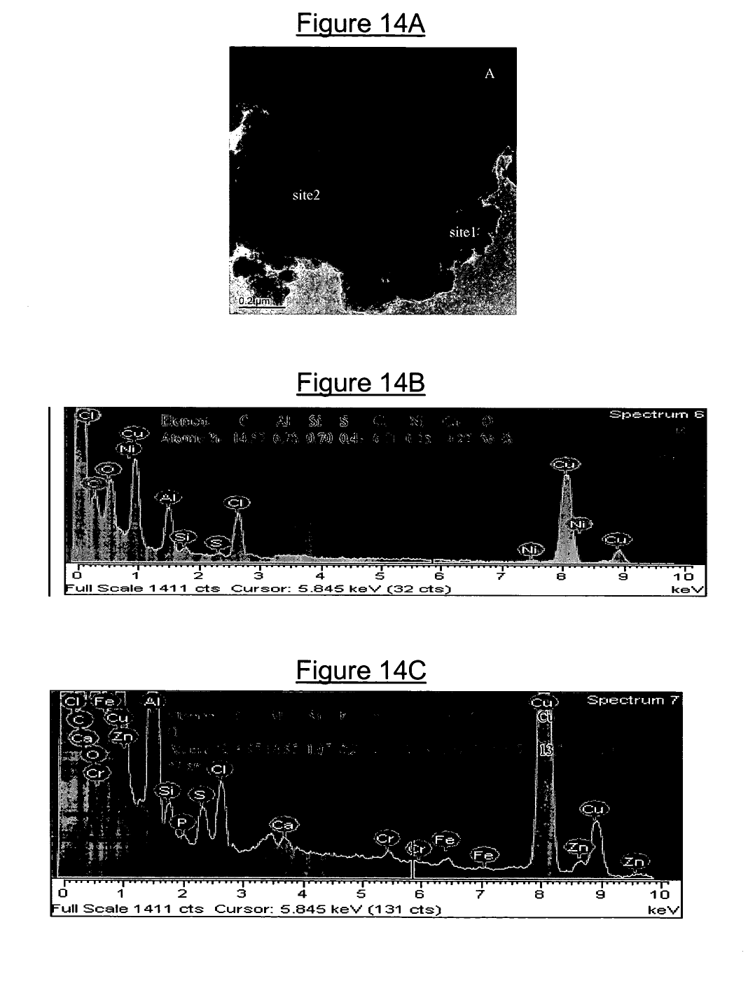

The crystalline structures obtained from the media containing APB for 2 weeks were analyzed. The results showed that trace elements, as well as the element Si, were deposited with crystalline structures (FIGS. 14A, B, C). The components from one crystalline structure in different loci showed different element distribution. The edge of the crystalline structure contained C, O, S, Cl, Si, Ni and Al (FIGS. 14A and B), while the center of the crystalline contained the same elements except Ni (FIGS. 14A and C). Some other components such as P, Ca, Cr, Fe and Zn were also detected in the center. The deposition/mineralization from APB alone in normal media is able to form the crystallines with trace elements and Si, which indicated that osteoblasts have no direct effect on the process.

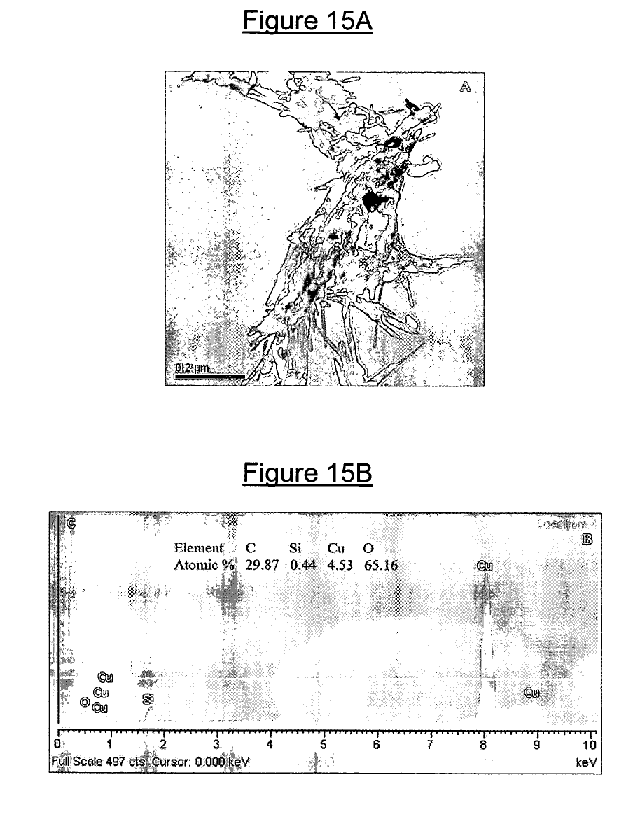

Non-osteoblast cells, instead of osteoblasts, were incubated with the APB to study the sequential order of deposits. Cr, Ni, and Si could also be detected in those crystalline structures formed in the media containing myoblasts and APB (FIGS. 15A, B). The results further confirmed that the process of deposition/mineralization was osteoblasts-independent.

Discussion

In this experiment, cellular mineral deposition was determined in natural anorganic bone scaffold. Under these conditions, spontaneous mineral depositions were detected in classical culture media without supplement. The osteoblastic cell culture system is considered as a valuable tool for investigation of the in vitro mineralization process as well as for evaluation of osteogenic cellular response to implant material. To provide useful information regarding the in vitro process of mineralization, the organic phosphate such as β-glycerolphosphate in bone culture systems was routinely used (Tenenbaum, H. C., J. Dent Res. 1981; 60: 1586-1589; Temembai, H. C., Heersche, J. N., Calcif. Tissue Int. 1982; 34: 76-79; Ecarot-Charrier, G., et al., J. Cell Biol. 1983; 96: 639-643; Robey, P. G., Termine, J. D, Calcif Tissue Int. 1985; 37: 453-460; Gotoh, Y., et al., Bone Miner. 1990; 8:239-250). Those experiments clearly indicated that organic phosphate was hydrolyzed by alkaline phosphatase to release free inorganic phosphate (Fortuna, R., et al., Calcif. Tissue Int. 1980; 30:217-225), thus proving the chemical potential for promoting mineralization. However, the natural deposition/mineralization is considered to be a very complicated system as natural bones have varying degrees of degradation owing to the process of osteoclasts; highly organized 3 dimensional structure; non-homogeneous component distribution and other effectors. The gradual dissolution of HA from natural bones releases calcium and phosphate ions that influences the nearby cell population and leads to a reprecipitation of calcium phosphates, thus enhancing the bone apposition and bonding to bony tissues (Daculsi, G., et al., Calcif. Tissue. Int. 1990; 46: 20-27; Bagambisa, F. B., et al., J. Biomed. Mater. Res. 1993; 27: 1047-1055). The dissolution characteristics imply that HA also served as a source of inorganic phosphate to enhance cell mineralization.

In this experiment, the dynamic anorganic bone provides not only basic constituents but also other trace/essential elements for the bone formation. The composition and crystal morphology of more than 40 deposits were determined by scanning electron microscopy (SEM) and energy dispersive X-ray analysis (EDX). The analysis revealed that all investigated deposits in the first week contained sodium, potassium, phosphate, sulfur, and calcium as major constituents. Within the major constituents, some samples were composted of other elements such as C, Cr, Si, and Mg. Most crystalline structures measured had a broad range of Ca/P molar ratio (between 0.5 to 2.7). The Ca/P measured from the control media showed a much-fixed ratio within 0.7 to 0.92. Once the deposits were taken from the media of the period of two-week mineralization, the major constituents diminished as new compositions of deposits were found. The major constituent was either element S or C with element Si. A variety of trace elements were found. The same phenomena was observed from the analysis of the media of APB alone or non-osteoblasts with APB. The examination of selected samples by means of SEM/EDX, revealed the characteristic morphology and elemental composition of the constituents of the variety of deposits. The morphology of crystalline structures showed a brunch-like shape, as well as other shapes such as a needle and crystal-like appearance, which are typical structures of silicate.

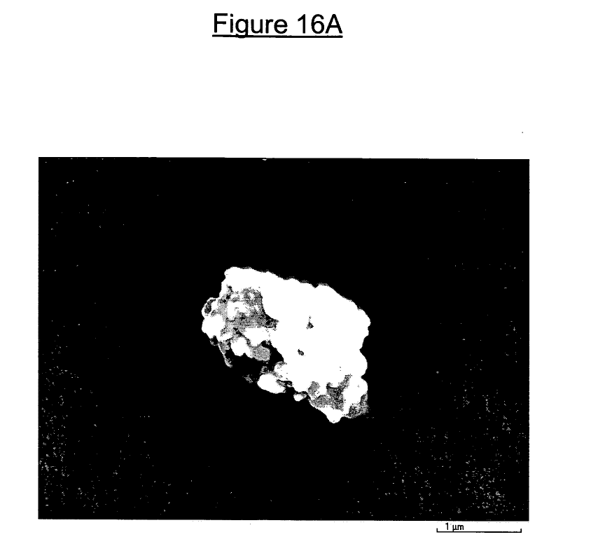

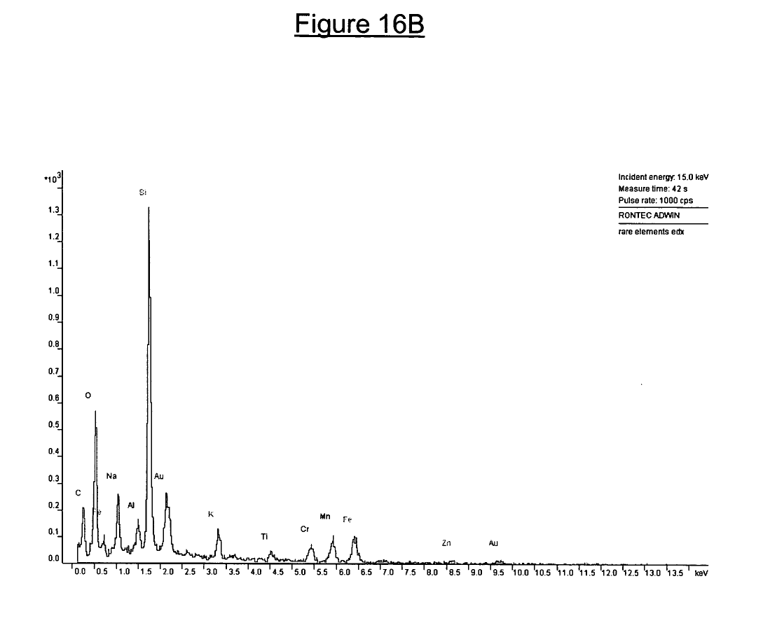

There are certain profiles shown in the deposition. Mg-containing deposits occasionally were observed in the early stage, completely absent in the later stage. The phosphate was deposited in the early phase and was hardly observed in the later stage. The carbonate deposit had a different profile compared to the phosphate-, Mg-containing deposits. The carbonate deposit was observed in the earlier stage and continued to precipitate with other constituents in the later phase. The silicon-containing deposits precipitated in the early stage and persistently occurred in the later stage of all deposits. Such a profile from silicon-containing deposits matched the trace element-containing deposits. In the early stages, elements Cr, and Co were found. A larger variety of trace elements were observed in the later stage. One typical precipitate from the media of osteoblasts with APB had a variety of trace elements with other components (C, O, Na and K, Al, Si), shown in FIGS. 16A, B. The size of this precipitate is around 2 μm. The reason why some elements were found in the early phase and completely undetectable in the later phase was due to the absolute occurrence in the early dissolution and/or the deposition to form a precipitate, which was partially supported by the precipitate observed. In the findings of Si and trace elements-containing deposits, it can be concluded that a sequential order of dissolution/precipitation of essential/trace elements during the process of bone formation occurred.

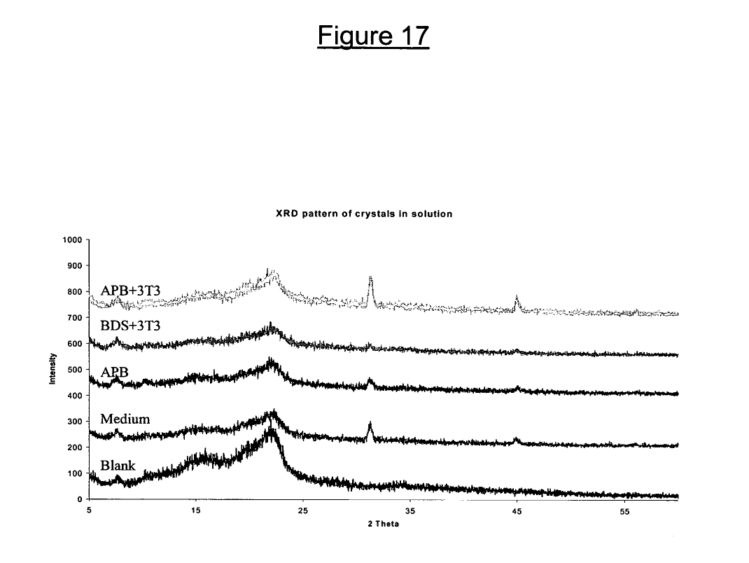

A further investigation was to check whether any crystal form could be detected, especially a precursor of HA or itself. It has been proposed that the biological apatite could form from several possible precursors such as amorphous calcium phosphate (ACP), brushit (DCPD), β-tricalcium phosphate (β-TCP) or octacalcium phosphate (OCP). (Brown, W. E., Chow, L. C., Ann. Res. Mater. Sci. 1976; 6: 213-226; Francis, M. D. and Webb, N. C., Calcify. Tissue Res. 1971; 6: 335-342). The XRD pattern analysis of several conditioned media from APB alone, APB with osteoblasts, and a synthetic calcium phosphate (BDS) with osteoblasts had a typical peak at 2θ of 31.4 and 45 (FIG. 17). As the crystal from the media containing osteoblasts with APB had the highest peaks, those peaks from the media itself or other conditioned media had either the second highest peak or almost no peak. Although these are characteristic peaks for NaCl, the peak representing calcium phosphate of 2θ is 32. Based on the height of the peak at 32 from the APB with osteoblasts, there is a high likelihood ofcalcium phosphate being present.

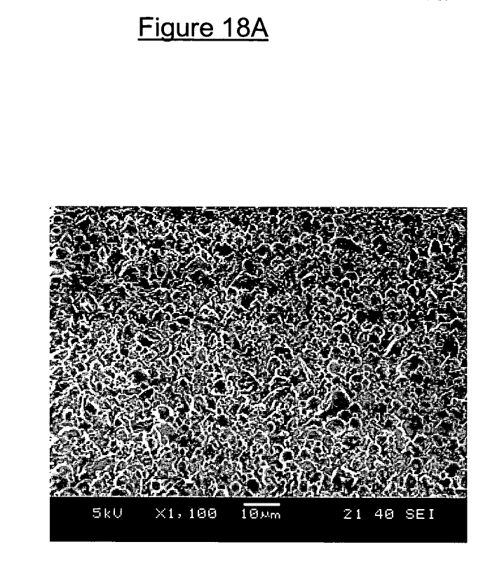

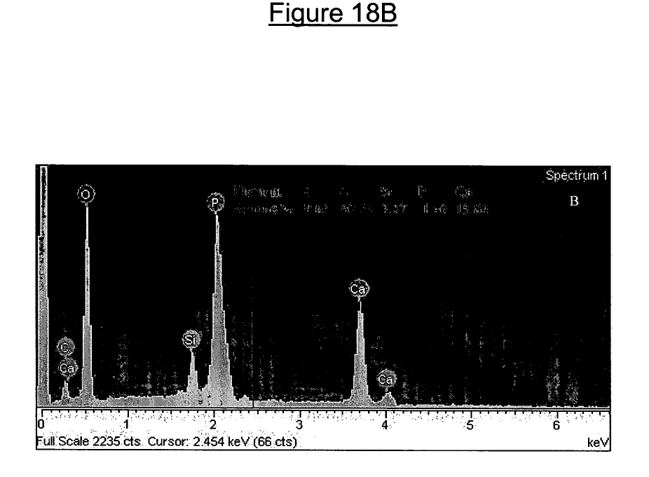

BDS is a commercial synthetic apatite. It has been used as a control while cells can proliferate well on the surface of such a scaffold. The component analysis of BDS by SEM-EDX showed elements C, O, Si, P, and Ca, which the Ca/P is 1.36 (FIGS. 18A, B). Although BDS contains element Si, no deposit with Si during the dissolution of BDS in the presence of cells (data not shown) could be found regardless of the high atomic percentage of Si (2.87%) in the APB. In Example 1, it has been shown that the main components of the APB are P, O, C, Ca, and Mg. No obvious Si as well as trace/essential elements could be detected under SEM-EDX. The result indicates that the Si in the BDS seems to be difficult to dissolve compared to that in the APB dose. Although the dissolution properties of the synthetic apatite is of interest in relation to the coupled demineralization and remineralization processes associated with dental caries (LeGeros, R. Z., Prog. Crystal Growth Charact. 1981; 4: 1-45; LeGeros, R Z., Suga, S., Calcif. Tissue Int. 1980; 32: 169-174; LeGeros, R Z., et al., Calc. Tiss. Res. 1978; 26:111-116; Boskey, A., Posner, A. S., J. Phys. Chem. 1976; 80: 40-45), it will be a great challenge to study the reactions of calcium compounds to form calcium deficient apatites (Zhuoer, H., J. analy. Atomic spectro. 1994; 9: 11-15; Mirtchi, A. A., et al., Biomater. 1990; 11: 83-88; LeGeros, R. Z., et al., J. Dent. Res. 1982; 61-342, Abstr. 1482) as trace elements and other essential elements like Si, Al, and Mg could play an important role in the dissolution/precipitation.

The study of in vivo dissolution/deposition is critical to understand the processes of bone remodeling. The bioactivity of biological anorganic apatites in the dissolution of calcium, phosphate, silicon, and other trace/essential elements provide the natural solubility in a biological environment. Our findings strengthened the theory that the dissolution/precipitation processes in biological systems appear to be dependent on their composition and microstructure in physiological conditions. Although the osteoblastic cells can highly differentiate through the incubation of APB, the differentiated cells have no influence on the process of dissolution/deposition. HA ceramics studies indicate that the bioactivity is closely related to the microstructure (Nelson, D. G., et al., J. Ultrastruct. Res. 1983; 84:1-15; Daculsi, G., et al., Calcif. Tissue Int. 1989; 45: 95-103). Silicon-substituted HA bioceramics have an increased rate of dissolution compared to the pure HA dose (Porter, A. E., et al., Biomater. 2003; 24: 4609-4620). The Si—, Mg— or Si-substitutes HA have a good biocompatibility and are considered better implant biomaterials (Carlisle, E. M., Science 1970; 167:279-).

1. A method for the preparation of an implantable biomaterial comprising the steps of:
a) obtaining bone tissue;
b) boiling the bone tissue;
c) treating the bone tissue to remove the collagen.

2. The method of claim 1, wherein after the treatment of step (c) no collagen is detectable by SEM-EDX.

3. The method of claim 1, wherein the boiling step is carried out in water.

4. The method of claim 3, wherein the boiling step is carried out in distilled water.

5. The method of claim 1, comprising cleaning the bone tissue before boiling.

6. The method of claim 5, wherein the bone tissue is cleaned mechanically, by air or by means of a liquid.

7. The method of claim 1, wherein the bone tissue is cleaned with water.

8. The method of claim 1, wherein the step c) comprises treating the bone tissue at a temperature of 200-250° C. to melt and denature the collagen, and further treating with a solvent to dissolve the collagen.

9. The method of claim 8, wherein the temperature is 210° C.

10. The method of claim 8, wherein the solvent to dissolve the collagen is ethanol, hydrazine, methanol, and/or guanidine hydrochloride.

11. The method of claim 10, further comprising treating the bone tissue with ultrasounds.

12. The method of claim 8, wherein after the treatment of step c) no collagen is detectable by SEM-EDX.

13. The method of claim 1, further comprising treating the bone tissue with ultrasounds.

14. The method of claim 1, further comprising a step d) of cutting the bone tissue into a predetermined shape.

15. The method of claim 14, wherein the bone tissue is cut up by means of a high pressure water jet.

16. The method of claim 14, further comprising a step e) of sterilising the cut bone before implant.

17. The method of claim 16, further comprising a step of packaging the implantable biomaterial.

18. The method of claim 16, further comprising the step of implanting the biomaterial in a vertebrate.

19. The method of claim 1, further comprising the step of implanting the biomaterial in a vertebrate.

20. The method of claim 1, further comprising a step of cultivating in vitro the implantable biomaterial prior to implantation.

21. The method of claim 1, further comprising a step of seeding the implantable biomaterial with the patient's own cells prior to implantation.

22. The method of claim 1, wherein the obtained implantable biomaterial has a Ca/P ratio of 1.64.

23. The method of claim 1, comprising the steps of:
obtaining bone tissue;
cleaning the bone tissue with water;
boiling the bone tissue in water;
cleaning the boiled bone tissue;
drying the bone tissue;
treating the bone tissue at a high temperature such to melt and denature collagen;
treating the bone tissue with a solvent and/or treating with ultrasounds to dissolve the collagen;
allowing the solvent to evaporate from the bone tissue;

24. The method of claim 1, wherein no collagen is detectable by SEM-EDX in the implantable biomaterail.

25. The method of claim 1, wherein the obtained implantable material is a anorganic bone.

26. The method of claim 1, wherein the obtained implantable material is free from organic matrix.

27. The method of claims 1, further comprising cutting the bone tissue into a predetermined shape, and sterilising the cut bone tissue before implanting.

28. The method of claim 1, wherein the method does not comprise a step of treating the bone tissue with a fluid in supercritical state.

29. An implantable biomaterial prepared according to the process of claim 1.

30. The implantable biomaterial of claim 29, wherein the implantable biomaterial has a Ca/P ratio of 1.64.

31. The implantable biomaterial of claim 29, wherein the implantable biomaterial is cultivated in vitro prior to implantation.

32. The implantable biomaterial of claim 29, wherein the implantable biomaterial is seeded with the patient's own cells prior to implantation.

33. The implantable material of claim 29, which is an anorganic bone free from organic matrix.

34. The implantable biomaterial of claim 29, wherein no collagen is detectable by SEM-EDX.

35. An implantable biomaterial, wherein the implantable biomaterial is made from bone tissue and has a Ca/P ratio of 1.64.

36. The implantable biomaterial of claim 35, wherein no collagen is detectable by SEM-EDX in the implantable biomaterial.
